SEQUENCE LISTING (10 sequences)

SEQ ID NO: 1 - protein, 107 aa, Artificial Sequence [sequence omitted]

SEQ ID NO: 2 - protein, 116 aa, Artificial Sequence [sequence omitted]

SEQ ID NO: 3 - protein, 214 aa, Artificial Sequence
  synthetic peptide
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 446 aa, Artificial Sequence
  synthetic peptide
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 108 aa, Artificial Sequence
  synthetic peptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 6 - protein, 121 aa, Artificial Sequence
  synthetic peptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 7 - protein, 215 aa, Artificial Sequence
  synthetic peptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 451 aa, Artificial Sequence
  synthetic peptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 10 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

(19) United States
(12) Patent Application Publication
(10) Pub. No.: US 2020/0062850 A1
(43) Pub. Date: Feb. 27, 2020

(54) COMBINATION OF AN ANTI-VEGFR-2 ANTIBODY AND AN ANTI-PD-L1 ANTIBODY FOR THE TREATMENT OF CANCER

(71) Applicants: ImClone LLC, Indianapolis, IN (US); MedImmune Ltd., Cambridge (GB)

(72) Inventors: Richard Brian GAYNOR, Carmel, IN (US); Maria KARASARIDES, Boston, MA (US)

(21) Appl. No.: 16/339,776

(22) PCT Filed: Oct. 27, 2017
(86) PCT No.: PCT/US2017/058684

§ 371 Date: Apr. 5, 2019

Related U.S. Application Data
(60) Provisional application No. 62/460,366, filed on Feb. 17, 2017.
(60) Provisional application No. 62/414,320, filed on Oct. 28, 2016.

(51) Int. Cl. C07K 16/28; C07K 16/30; A61P 35/00

(52) U.S. Cl. CPC C07K 16/2863; C07K 16/2827; C07K 16/3046; C07K 2317/76; C07K 2317/21; C07K 2317/24; C07K 2317/73; A61P 35/00

(57) ABSTRACT

The present disclosure relates to a combination of anti-human VEGFR-2 antibodies (Ramucirumab) and anti-human PD-L1 (durvalumab) antibodies for treating certain disorders, including advanced gastric or gastroesophageal junction adenocarcinomas, non-small cell lung cancer, and hepatocellular carcinomas. Sequences 3 & 4 represent Ramucirumab VL and VH; Sequences 7 & 8 those of Durvalumab.

The present invention relates to a combination of an anti-human VEGFR-2 antibody and an anti-human PD-L1 antibody, and to methods of using said combination to treat certain disorders, such as locally advanced and unresectable or metastatic gastrointestinal or thoracic malignancies.

Tumor growth often coincides with angiogenesis and immunosuppression. Programmed death receptor-1 (programmed death-1 or PD-1) can be expressed on the cell surface of activated T-cells under healthy conditions. Engagement of PD-1 via its ligands, Programmed death ligand-1 (PD-L1) or Programmed death ligand-2 (PD-L2), is known to down regulate immune responses, including anti-tumor immune responses. In this regard, PD-L1 expression is known to lead to the suppression of T-cell migration, proliferation and secretion of cytotoxic mediators, and restricts tumor cell killing. Anti-VEGFR-2 (vascular endothelial growth factor receptor-2) antibodies have been shown to improve T cell infiltration into tumors and inhibit migration of tumor associated macrophages in preclinical studies.

Ramucirumab (Cyramza®) is a human IgG1 monoclonal antibody directed against human vascular endothelial growth factor receptor 2 (VEGFR-2). Ramucirumab and methods of making and using ramucirumab are disclosed in WO2003/075840. Ramucirumab is approved by the United States Food and Drug Administration as a single agent or in combination with paclitaxel, for the treatment of advanced gastric or gastroesophageal junction adenocarcinoma, with disease progression on or after prior fluoropyrimidine- or platinum-containing chemotherapy; in combination with docetaxel, for the treatment of metastatic non-small cell lung cancer with disease progression on or after platinum-based chemotherapy. Patients with epidermal growth factor receptor (EGFR) or anaplastic lymphoma kinase (ALK) genomic tumor aberrations should have disease progression on FDA-approved therapy for these aberrations prior to receiving CYRAMZA®; and in combination with FOLFIRI (irinotecan, folinic acid, and 5-fluorouracil), for the treatment of metastatic colorectal cancer with disease progression on or after prior therapy with bevacizumab, oxaliplatin, and a fluoropyrimidine.

Durvalumab is an investigational human monoclonal antibody directed against human PD-L1. Durvalumab is currently being investigated in an extensive clinical trial program.

Often, patients with gastric or esophageal cancers are diagnosed at an advanced stage when surgical treatment is no longer available. While chemotherapy remains a mode of treatment for those afflicted with these advanced diseases, those treated with chemotherapy as a first line often have a median survival of less than one year. While ramucirumab therapy may provide an additive benefit when used in combination with chemotherapy for treating advanced gastric or gastro-esophageal junction adenocarcinomas, a significant fraction of these patients do not live for more than two years. Thus, there exists a need for improved therapies to treat locally advanced and unresectable or metastatic gastrointestinal or thoracic malignancies.

The present invention is derived from the ongoing Phase Ia clinical trial of the combination of ramucirumab and durvalumab (“Phase 1 study of ramucirumab (R) plus durvalumab (D) in patients (pts) for treating locally advanced and unresectable or metastatic gastrointestinal or thoracic malignancies (NCT02572687 (the “Study”)). Surprisingly, the present invention discloses the combination of an anti-human VEGFR-2 antibody and an anti-human PD-L1 antibody as part of an effective and improved treatment regimen for advanced gastric or gastroesophageal junction adenocarcinomas. Surprisingly, the present invention also discloses the combination of ramucirumab and durvalumab as part of an effective and improved treatment regimen for advanced gastric or gastroesophageal junction adenocarcinomas.

As used herein, the term “human VEGFR-2” refers to Human Vascular Endothelial Growth Factor Receptor 2, having the amino acid sequence of SEQ ID NO: 9. VEGFR-2 is also known as KDR.

As used herein, the term “human PD-L1” refers to Human Programmed Death Receptor Ligand One, having the amino acid sequence of SEQ ID NO: 10.

Ramucirumab is also known as CYRAMZA® and has the CAS registry number 947687-13-0. Ramucirumab is an anti-human VEGFR-2 antibody comprising two light chains, each of the light chains having the amino acid sequence of SEQ ID NO: 3, and two heavy chains, each of the heavy chains having the amino acid sequence of SEQ ID NO: 4. The light chain variable region of ramucirumab is that given in SEQ ID NO: 1. The heavy chain variable region of ramucirumab is that given in SEQ ID NO: 2.

The anti-human VEGFR-2 antibody selected will have a sufficiently strong binding affinity for human VEGFR-2. For example, the antibody will generally bind VEGFR-2 with a K_{d} value of between about 100 nM and about 1 pM. Antibody affinities may be determined by a surface plasmon resonance based assay (such as the BIAcore assay is described in WO2005/012359); enzyme-linked immunosorbent assay (ELISA); and competition assays (e.g. a radiolabeled antigen binding assay (RIA)), for example. In one embodiment, Kd is measured by a RIA performed with ramucirumab.

Durvalumab is an anti-human PD-L1 antibody that comprises two light chains and two heavy chains, and each of the light chains comprise the amino acid sequence of SEQ ID NO: 7 and each of the heavy chains comprise the amino acid sequence of SEQ ID NO: 8. The light chain variable region of durvalumab is that given in SEQ ID NO: 5. The heavy chain variable region of durvalumab is that given in SEQ ID NO: 6. Durvalumab has been previously described (see, for example, World Health Organization (2014) and the “International Nonproprietary Names for Pharmaceutical Substances (INN), Proposed INN: List 112” WHO Drug Information 28 (4), pages 496-497). Durvalumab has a CAS Registration Number of 1428935-60-7.

Unless indicated otherwise, the term “antibody” refers to an immunoglobulin molecule comprising two heavy chains (HC) and two light chains (LC) interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “light chain variable region” or “LCVR” refers to a portion of a light chain of an antibody molecule that includes the amino acid sequences of CDRs and framework regions (FRs).

As used herein, the term “heavy chain variable region” “HCVR” refers to a portion of a heavy chain of an antibody molecule that includes the amino acid sequences of CDRs and FRs.

As used herein, the term “kit” refers to a package comprising at least two separate containers, wherein a first container contains an anti-human VEGFR-2 antibody, and a second container contains an anti-human PD-L1 antibody. In some examples, the first container comprises ramucirumab and the second container comprises durvalumab. A “kit” may also include instructions to administer all or a portion of the contents of these first and second containers to a cancer patient, preferably an advanced gastric or gastroesophageal junction adenocarcinoma patient.

As used herein, the terms “treating,” “treat,” or “treatment” refer to restraining, slowing, lessening, reducing, or reversing the progression or severity of an existing symptom, disorder, condition, or disease, or ameliorating clinical symptoms of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Treatment can also mean prolonging survival as compared to expected survival if not receiving treatment. Those in need of treatment include those already with the disease. In some examples, the present invention can be used as a medicament.

As used herein, the term “patient” refers to a mammal, preferably a human.

As used herein, the term “cancer” refers to or describes the physiological condition in patients that is typically characterized by unregulated cellular proliferation. Included in this definition are benign and malignant cancers.

As used herein, the term “effective amount” refers to the amount or dose of anti-human VEGFR-2 antibody or an anti-human PD-L1 antibody, preferably ramucirumab or durvalumab, which provides an effective response in the patient under diagnosis or treatment.

As used herein, the term “effective response” of a patient or a patient's “responsiveness” to treatment with a combination of agents refers to the clinical or therapeutic benefit imparted to a patient upon administration of an anti-human VEGFR-2 antibody and an anti-human PD-L1 antibody, preferably ramucirumab and durvalumab.

Generally, dosage regimens may be adjusted to provide the optimum desired response (e.g., a therapeutic response). Treatment dosages may be titrated using routine methods known to those of skill in the art to optimize safety and efficacy. Dosing schedules will typically range from a single bolus dosage or continuous infusion, to multiple administrations per day (e.g., every 4-6 hours), or as indicated by the treating physician and the patient's condition. Dosing frequencies of the antibody will be determined by the physicians treating the patient and may be given daily, three times per week, weekly, every three weeks, or less often, and more preferably every two weeks. Dosing amounts of the antibodies will also be determined by the physicians treating the patient and may fall within customary ranges.

In some instances, dosage levels below the lower limit of the aforesaid dosing for the antibodies described herein may be more than adequate, while in other cases larger doses may be employed with acceptable side effects, and therefore the above dosage amount is not intended to limit the scope of the invention in any way.

The anti-human VEGFR-2 antibody may be administered from 2 to 20 mg/kg, weekly, every two weeks, or every three weeks, depending on tumor type, and patient factors. Preferably, ramucirumab may be administered at 8 mg/kg intravenously every two weeks starting on day 1 of a 21-day cycle.

The anti-human PD-L1 antibody may be administered from 750 mg every two weeks, or every three weeks, depending on tumor type, and patient factors. Preferably, durvalumab may be administered at 750 mg intravenously every two weeks starting on day 1 of a 28-day cycle.

Ramucirumab may be administered from 2 to 20 mg/kg, weekly, every two weeks, or every three weeks, depending on tumor type, and patient factors. Preferably, ramucirumab may be administered at 8 mg/kg intravenously every two weeks starting on day 1 of a 21-day cycle.

Durvalumab may be administered from 750 mg every two weeks, or every two weeks, depending on tumor type, and patient factors. Preferably, durvalumab may be administered at 750 mg intravenously every two weeks starting on day 1 of a 28-day cycle. The route of administration may be varied in any way, limited by the physical properties of the drugs and the convenience of the patient and the caregiver. Preferably, ramucirumab and durvalumab are formulated for parenteral administration, such as intravenous or subcutaneous administration.

As used herein, the phrase “in combination with” refers to the administration of an anti-human VEGFR-2 antibody and an anti-human PD-L1 antibody, preferably ramucirumab and durvalumab.

The therapeutically effective amount of the treatment of the invention can be measured by various endpoints commonly used in evaluating cancer treatments, including, but not limited to: extending survival (including OS and PFS); resulting in an objective response (including a CR or a PR); tumor regression, tumor weight or size shrinkage, longer time to disease progression, increased duration of survival, longer PFS, improved OS rate, increased duration of response, and improved quality of life and/or improving signs or symptoms of cancer.

As used herein, the term “progressive disease” (PD) refers to least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “partial response,” (PR) refers to at least a 30% decrease in the sum of diameters of target lesions, taking as reference the baseline sum diameters.

As used herein, the term “complete response” (CR) refers to the disappearance of all target lesions with the short axes of any target lymph nodes reduced to <10 mm.

As used herein, the term “stable disease” (SD) refers to neither sufficient shrinkage to qualify for PR nor sufficient increase to qualify for PD, taking as reference the smallest sum of diameters while on study.

As used herein, the term “not evaluable” (NE) refers to when an incomplete radiologic assessment of target lesions is performed or there is a change in the method of measurement from baseline that impacts the ability to make a reliable evaluation of response.

As used herein, the term “objective response rate” (ORR) is equal to the proportion of patients achieving a best overall response of partial or complete response (PR+CR) according to RECIST 1.1.

As used herein, the term “overall survival” (OS) refers to the percentage of patients remaining alive for a defined period of time, such as 1 year, 5 years, etc. from the time of diagnosis or treatment. In a preferred embodiment, OS refers to the time from the date of randomization in the Study to the date of death from any cause. If the patient is alive at the end of the follow-up period or is lost to follow-up, OS data is censored on the last date the patient is known to be alive. Overall survival is evaluated by the Kaplan-Meier method, and a 95% confidence interval (CI) is provided for the median OS in each treatment arm.

As used herein, the term “progression-free survival” (PFS) refers to the patient remaining alive without the cancer progressing or getting worse. In a preferred aspect of the invention, PFS is defined as the time from randomization in the Study until the first radiographic documentation of objective progression as defined by RECIST (Version 1.1), or death from any cause. Patients who die without a reported prior progression will be considered to have progressed on the day of their death. Patients who did not progress or are lost to follow-up will be censored at the day of their last radiographic tumor assessment.

As used herein, the term “disease control rate” (DCR) refers to lack of disease progression and rate thereof. It refers to the group of patients with a best overall response categorized as CR, PR or SD (specifically excluding the patients with PD), wherein the best overall response is the best response recorded from the start of treatment until PD.

As used herein, the term “clinical benefit rate,” refers to SD or better at 12 weeks. The tumor response rate of SD or better (i.e. CR+PR+SD) at 12 weeks is defined as the proportion of patients with a response of SD or better, as defined by RECIST 1.1, at 12 weeks following the first dose of study therapy. Patients will be considered “failure” if they die or if radiographic evaluation indicates a response of PD at 12 weeks or before.

As used herein, the term “extending survival” is meant as increasing OS or PFS in a treated patient relative to i) an untreated patient, ii) a patient treated with less than all of the anti-tumor agents in a particular combination therapy, or iii) a control treatment protocol. Survival is monitored following the initiation of treatment or following the initial diagnosis of cancer.

As used herein, the term “best overall response” is the best response recorded from the start of the study treatment until the earliest of objective progression or start of new anticancer therapy, taking into account any requirement for confirmation. The patient's best overall response assignment will depend on the findings of both target and nontarget disease and will also take into consideration the appearance of new lesions. The best overall response will be calculated via an algorithm using the assessment responses provided by the investigator over the course of the trial.

A method of treating advanced gastric or gastroesophageal junction adenocarcinoma, comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6.

A method of treating advanced gastric or gastroesophageal junction adenocarcinoma, comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein the anti-human VEGFR-2 antibody further comprises a light chain having the amino acid sequence of SEQ ID NO: 3 and a heavy chain having the amino acid sequence of SEQ ID NO: 4; optionally, wherein the anti-human PD-L1 antibody further comprises a light chain having the amino acid sequence of SEQ ID NO: 7 and a heavy chain having the amino acid sequence of SEQ ID NO: 8.

A method of treating advanced gastric or gastroesophageal junction adenocarcinoma, comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein the anti-human VEGFR-2 antibody further comprises a light chain having the amino acid sequence of SEQ ID NO: 3 and a heavy chain having the amino acid sequence of SEQ ID NO: 4, and the anti-human PD-L1 antibody further comprises a light chain having the amino acid sequence of SEQ ID NO: 7 and a heavy chain having the amino acid sequence of SEQ ID NO: 8.

A method of treating advanced gastric or gastroesophageal junction adenocarcinoma, comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks.

A method of treating advanced gastric or gastroesophageal junction adenocarcinoma, comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein the anti-human PD-L1 antibody is administered at a dose of 750 mg every two weeks.

A method of treating advanced gastric or gastroesophageal junction adenocarcinoma, comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two week, and the anti-human PD-L1 antibody is administered at a dose of 750 mg every two weeks.

A method of treating advanced gastric or gastroesophageal junction adenocarcinoma, comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein the anti-human VEGFR-2 antibody is ramucirumab.

A method of treating advanced gastric or gastroesophageal junction adenocarcinoma, comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein the anti-human PD-L1 antibody is durvalumab.

A method of treating advanced gastric or gastroesophageal junction adenocarcinoma, comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein the anti-human VEGFR-2 antibody is ramucirumab and the the anti-human PD-L1 antibody is durvalumab.

A kit for the treatment of advanced gastric or gastroesophageal junction adenocarcinoma, the kit comprising an anti-human VEGFR-2 (SEQ ID NO: 9) antibody comprising a light chain having the amino acid sequence of SEQ ID NO: 3 and a heavy chain having the amino acid sequence of SEQ ID NO: 4 and an anti-human PD-L1 (SEQ ID NO: 10) antibody comprising a light chain having the amino acid sequence of SEQ ID NO: 7 and a heavy chain having the amino acid sequence of SEQ ID NO: 8.

A kit for the treatment of advanced gastric or gastroesophageal junction adenocarcinoma, the kit comprising a first pharmaceutical composition comprising an anti-human VEGFR-2 (SEQ ID NO: 9) antibody comprising a light chain having the amino acid sequence of SEQ ID NO: 3 and a heavy chain having the amino acid sequence of SEQ ID NO: 4 and a second pharmaceutical composition comprising an anti-human PD-L1 (SEQ ID NO: 10) antibody comprising a light chain having the amino acid sequence of SEQ ID NO: 7 and a heavy chain having the amino acid sequence of SEQ ID NO: 8.

A kit for the treatment of advanced gastric or gastroesophageal junction adenocarcinoma, the kit comprising an anti-human VEGFR-2 (SEQ ID NO: 9) antibody comprising a light chain having the amino acid sequence of SEQ ID NO: 3 and a heavy chain having the amino acid sequence of SEQ ID NO: 4 and an anti-human PD-L1 (SEQ ID NO: 10) antibody comprising a light chain having the amino acid sequence of SEQ ID NO: 7 and a heavy chain having the amino acid sequence of SEQ ID NO: 8; wherein the anti-human VEGFR-2 antibody is ramucirumab and the anti-human PD-L1 antibody is durvalumab.

A kit for the treatment of advanced gastric or gastroesophageal junction adenocarcinoma, the kit comprising an anti-human VEGFR-2 (SEQ ID NO: 9) antibody comprising a light chain having the amino acid sequence of SEQ ID NO: 3 and a heavy chain having the amino acid sequence of SEQ ID NO: 4 and an anti-human PD-L1 (SEQ ID NO: 10) antibody comprising a light chain having the amino acid sequence of SEQ ID NO: 7 and a heavy chain having the amino acid sequence of SEQ ID NO: 8; wherein the first pharmaceutical composition further comprises one or more pharmaceutically acceptable carriers, diluents, or excipients, and the second pharmaceutical composition further comprises one or more pharmaceutically acceptable carriers, diluents or excipients.

An anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6.

An anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody further comprises a light chain having the amino acid sequence of SEQ ID NO: 3 and a heavy chain having the amino acid sequence of SEQ ID NO: 4.

An anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human PD-L1 antibody further comprises a light chain having the amino acid sequence of SEQ ID NO: 7 and a heavy chain having the amino acid sequence of SEQ ID NO: 8.

An anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody further comprises a light chain having the amino acid sequence of SEQ ID NO: 3 and a heavy chain having the amino acid sequence of SEQ ID NO: 4, and the anti-human PD-L1 antibody further comprises a light chain having the amino acid sequence of SEQ ID NO: 7 and a heavy chain having the amino acid sequence of SEQ ID NO: 8.

An anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks.

An anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human PD-L1 antibody is administered at a dose of 750 mg every two weeks.

An anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks, and the anti-human PD-L1 antibody is administered at a dose of 750 mg every two weeks.

An anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; optionally, wherein the anti-human VEGFR-2 antibody is administered at a dose of 8 mg/kg every two weeks, and the anti-human PD-L1 antibody is administered at a dose of 750 mg every two weeks; wherein the anti-human VEGFR-2 antibody is ramucirumab and the anti-human PD-L1 antibody is durvalumab.

An anti-human VEGFR-2 (SEQ ID NO: 9) antibody and an anti-human PD-L1 (SEQ ID NO: 10) antibody for the manufacture of a medicament for the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6.

An anti-human VEGFR-2 (SEQ ID NO: 9) antibody and an anti-human PD-L1 (SEQ ID NO: 10) antibody for the manufacture of a medicament for the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody further comprises a light chain having the amino acid sequence of SEQ ID NO: 3 and a heavy chain having the amino acid sequence of SEQ ID NO: 4.

An anti-human VEGFR-2 (SEQ ID NO: 9) antibody and an anti-human PD-L1 (SEQ ID NO: 10) antibody for the manufacture of a medicament for the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6; wherein the anti-human PD-L1 antibody further comprises a light chain having the amino acid sequence of SEQ ID NO: 7 and a heavy chain having the amino acid sequence of SEQ ID NO: 8.

An anti-human VEGFR-2 (SEQ ID NO: 9) antibody and an anti-human PD-L1 (SEQ ID NO: 10) antibody for the manufacture of a medicament for the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody further comprises a light chain having the amino acid sequence of SEQ ID NO: 3 and a heavy chain having the amino acid sequence of SEQ ID NO: 4, and the anti-human PD-L1 antibody further comprises a light chain having the amino acid sequence of SEQ ID NO: 7 and a heavy chain having the amino acid sequence of SEQ ID NO: 8.

An anti-human VEGFR-2 (SEQ ID NO: 9) antibody and an anti-human PD-L1 (SEQ ID NO: 10) antibody for the manufacture of a medicament for the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks, and the anti-human PD-L1 antibody is administered at a dose of 750 mg every two weeks.

An anti-human VEGFR-2 (SEQ ID NO: 9) antibody and an anti-human PD-L1 (SEQ ID NO: 10) antibody for the manufacture of a medicament for the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6; optionally, wherein the anti-human VEGFR-2 antibody is administered at a dose of 8 mg/kg every two weeks, and the anti-human PD-L1 antibody is administered at a dose of 750 mg every two weeks; wherein the anti-human VEGFR-2 antibody is ramucirumab, and the anti-human PD-L1 antibody is durvalumab

A first pharmaceutical composition comprising an anti-human VEGFR-2 (SEQ ID NO: 9) antibody for use in simultaneous, separate, or sequential combination with a second pharmaceutical composition comprising an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6.

A first pharmaceutical composition comprising an anti-human VEGFR-2 (SEQ ID NO: 9) antibody for use in simultaneous, separate, or sequential combination with a second pharmaceutical composition comprising an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody further comprises a light chain having the amino acid sequence of SEQ ID NO: 3 and a heavy chain having the amino acid sequence of SEQ ID NO: 4 and the anti-human PD-L1 antibody further comprises a light chain having the amino acid sequence of SEQ ID NO: 7 and a heavy chain having the amino acid sequence of SEQ ID NO: 8.

A first pharmaceutical composition comprising an anti-human VEGFR-2 (SEQ ID NO: 9) antibody for use in simultaneous, separate, or sequential combination with a second pharmaceutical composition comprising an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks and the anti-human PD-L1 antibody is administered at a dose of 750 mg every two weeks.

A first pharmaceutical composition comprising an anti-human VEGFR-2 (SEQ ID NO: 9) antibody for use in simultaneous, separate, or sequential combination with a second pharmaceutical composition comprising an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; optionally, wherein the anti-human VEGFR-2 antibody is administered at a dose of 8 mg/kg every two weeks and the anti-human PD-L1 antibody is administered at a dose of 750 mg every two weeks; wherein the anti-human VEGFR-2 antibody is ramucirumab, and the anti-human PD-L1 antibody is durvalumab.

In some embodiments, the invention includes a method of treating advanced gastric or gastroesophageal junction adenocarcinoma, non-small cell lung cancer, or hepatocellular carcinoma comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein (a) the anti-human VEGFR-2 antibody is administered at a dose of 8 mg/kg every two weeks and the anti-human PD-L1 antibody is administered at a dose of 750 mg every two weeks when advanced gastric or gastroesophageal junction adenocarcinoma or hepatocellular carcinoma is treated, or (b) the anti-human VEGFR-2 antibody is administered at a dose of 10 mg/kg every three weeks and the anti-human PD-L1 antibody is administered at a dose of 1125 mg every three weeks when non-small cell lung cancer is treated.

In some embodiments, the invention includes a method of treating advanced gastric or gastroesophageal junction adenocarcinoma comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein (a) the anti-human VEGFR-2 antibody is administered at a dose of 8 mg/kg every two weeks and the anti-human PD-L1 antibody is administered at a dose of 750 mg every two weeks.

In some embodiments, the invention includes: an anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, non-small cell lung cancer, or hepatocellular carcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein (a) the anti-human VEGFR-2 antibody is administered at a dose of 8 mg/kg every two weeks and the anti-human PD-L1 antibody is administered at a dose of 750 mg every two weeks when advanced gastric or gastroesophageal junction adenocarcinoma or hepatocellular carcinoma is treated, or (b) the anti-human VEGFR-2 antibody is administered at a dose of 10 mg/kg every three weeks and the anti-human PD-L1 antibody is administered at a dose of 1125 mg every three weeks when non-small cell lung cancer is treated.

In some embodiments, the invention includes: an anti-human VEGFR-2 (SEQ ID NO: 9) antibody for use in simultaneous, separate, or sequential combination with an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks and the anti-human PD-L1 antibody is administered at a dose of 750 mg every two weeks when advanced gastric or gastroesophageal junction adenocarcinoma.

In some embodiments, the invention includes: an anti-human VEGFR-2 (SEQ ID NO: 9) antibody for use in simultaneous, separate, or sequential combination with an anti-human PD-L1 (SEQ ID NO: 10) antibody, in the treatment of non-small cell lung cancer, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 10 mg/kg every three weeks and the anti-human PD-L1 antibody is administered at a dose of 1125 mg every three weeks.

In some embodiments, the invention includes: an anti-human VEGFR-2 (SEQ ID NO: 9) antibody for use in simultaneous, separate, or sequential combination with an anti-human PD-L1 (SEQ ID NO: 10) antibody, in the treatment of hepatocellular carcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks and the anti-human PD-L1 antibody is administered at a dose of 750 mg every two weeks.

In some embodiments, the invention includes: an anti-human VEGFR-2 (SEQ ID NO: 9) antibody and an anti-human PD-L1 (SEQ ID NO: 10) antibody for the manufacture of a medicament for the treatment of advanced gastric or gastroesophageal junction adenocarcinoma, non-small cell lung cancer, or hepatocellular carcinoma, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6; wherein (a) the anti-human VEGFR-2 antibody is administered at a dose of 8 mg/kg every two weeks and the anti-human PD-L1 antibody is administered at a dose of 750 mg every two weeks when advanced gastric or gastroesophageal junction adenocarcinoma or hepatocellular carcinoma is treated, or (b) the anti-human VEGFR-2 antibody is administered at a dose of 10 mg/kg every three weeks and the anti-human PD-L1 antibody is administered at a dose of 1125 mg every three weeks when non-small cell lung cancer is treated.

In some embodiments, the invention includes: an anti-human VEGFR-2 (SEQ ID NO: 9) antibody and an anti-human PD-L1 (SEQ ID NO: 10) antibody for the manufacture of a medicament for the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks and the anti-human PD-L1 antibody is administered at a dose of 750 mg every two weeks when advanced gastric or gastroesophageal junction adenocarcinoma or hepatocellular carcinoma is treated.

In some embodiments, the invention includes: an anti-human VEGFR-2 (SEQ ID NO: 9) antibody and an anti-human PD-L1 (SEQ ID NO: 10) antibody for the manufacture of a medicament for the treatment of non-small cell lung cancer, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 10 mg/kg every three weeks and the anti-human PD-L1 antibody is administered at a dose of 1125 mg every three weeks.

In some embodiments, the invention includes: an anti-human VEGFR-2 (SEQ ID NO: 9) antibody and an anti-human PD-L1 (SEQ ID NO: 10) antibody for the manufacture of a medicament for the treatment of hepatocellular carcinoma, wherein the anti-human VEGFR-2 antibody comprises a light chain variable region having the amino acid sequence of SEQ ID NO: 1 and a heavy chain variable region having the amino acid sequence of SEQ ID NO: 2, and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks and the anti-human PD-L1 antibody is administered at a dose of 750 mg every two weeks.

In some embodiments, the invention includes: a pharmaceutical composition comprising an anti-human VEGFR-2 (SEQ ID NO: 9) antibody for use in simultaneous, separate, or sequential combination with a pharmaceutical composition comprising an anti-human PD-L1 (SEQ ID NO: 10) antibody, in the treatment of advanced gastric or gastroesophageal junction adenocarcinoma, non-small cell lung cancer, or hepatocellular carcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein (a) the anti-human VEGFR-2 antibody is administered at a dose of 8 mg/kg every two weeks and the anti-human PD-L1 antibody is administered at a dose of 750 mg every two weeks when advanced gastric or gastroesophageal junction adenocarcinoma or hepatocellular carcinoma is treated, or (b) the anti-human VEGFR-2 antibody is administered at a dose of 10 mg/kg every three weeks and the anti-human PD-L1 antibody is administered at a dose of 1125 mg every three weeks when non-small cell lung cancer is treated.

In some embodiments, the invention includes: a pharmaceutical composition comprising an anti-human VEGFR-2 (SEQ ID NO: 9) antibody for use in simultaneous, separate, or sequential combination with a pharmaceutical composition comprising an anti-human PD-L1 (SEQ ID NO: 10) antibody, in the treatment of advanced gastric or gastroesophageal junction adenocarcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks and the anti-human PD-L1 antibody is administered at a dose of 750 mg every two weeks.

In some embodiments, the invention includes: a pharmaceutical composition comprising an anti-human VEGFR-2 (SEQ ID NO: 9) antibody for use in simultaneous, separate, or sequential combination with a pharmaceutical composition comprising an anti-human PD-L1 (SEQ ID NO: 10) antibody, in the treatment of non-small cell lung cancer, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 10 mg/kg every three weeks and the anti-human PD-L1 antibody is administered at a dose of 1125 mg every three weeks.

In some embodiments, the invention includes: a pharmaceutical composition comprising an anti-human VEGFR-2 (SEQ ID NO: 9) antibody for use in simultaneous, separate, or sequential combination with a pharmaceutical composition comprising an anti-human PD-L1 (SEQ ID NO: 10) antibody, in the treatment of hepatocellular carcinoma, wherein the anti-human VEGFR-2 antibody comprises a light chain variable region having the amino acid sequence of SEQ ID NO: 1 and a heavy chain variable region having the amino acid sequence of SEQ ID NO: 2 and the anti-human PD-L1 antibody comprises a light chain variable region having the amino acid sequence of SEQ ID NO: 5 and a heavy chain variable region having the amino acid sequence of SEQ ID NO: 6; wherein the anti-human VEGFR-2 antibody is administered at a dose of 8 mg/kg every two weeks and the anti-human PD-L1 antibody is administered at a dose of 750 mg every two weeks.

The antibodies described herein may readily be produced in mammalian cells, non-limiting examples of which includes CHO, NS0, HEK293 or COS cells. The host cells are cultured using techniques well known in the art. In this regard, an appropriate host cell can be either transiently or stably transfected with an expression system for secreting antibodies using an optimal predetermined HC:LC vector ratio or a single vector system encoding both HC (heavy chain) and LC (light chain). The vectors containing the polynucleotide sequences of interest (e.g., the polynucleotides encoding the polypeptides of the antibody and expression control sequences) can be transferred into the host cell by well-known methods, which may vary depending on the type of cellular host. Clarified media, into which the antibody has been secreted, may be purified using any of many commonly-used techniques. Various methods of protein purification may be employed and such methods are known in the art and described, for example, in Deutscher, Methods in Enzymology 182: 83-89 (1990) and Scopes, Protein Purification: Principles and Practice, 3rd Edition, Springer, NY (1994). In some examples, the medium may be conveniently applied to a column that has been equilibrated with a compatible buffer. The column may be washed to remove nonspecific binding components. The bound antibody may be eluted, for example, by pH gradient. Antibody fractions may be detected, such as by UV absorbance or SDS-PAGE, and then may be pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purity of the antibody after these chromatography steps is typically greater than 95%. The product may be immediately frozen at −70° C. or may be lyophilized. The following illustrates the unexpected improvement of patients treated with the combination of ramucirumab and durvalumab.

Phase 1 Study of Ramucirumab (R) Plus Durvalumab (D) in Patients (Pts) with Locally Advanced and Unresectable or Metastatic Gastrointestinal or Thoracic Malignancies (NCT02572687)

Eligible patients with advanced non-small cell lung cancer (NSCLC), advanced gastric or gastroesophageal junction adenocarcinoma (G/GEJ), or hepatocellular carcinoma (HCC) who progressed on prior systemic therapy were enrolled. Two dosing schedules will be evaluated in the Phase 1a/dose limiting toxicity (DLT) observation phase following 6+3 design: ramucirumab (10 mg/kg intravenous (IV)) and durvalumab (1125 mg IV) Q3W (21-day cycle) for NSCLC, and ramucirumab (8 mg/kg IV) and durvalumab (750 mg IV) Q2W (28-day cycle) for G/GEJ and HCC. After phase 1a, tumor cohorts were expanded to 20 patients who will receive study treatment until confirmed disease progression or unacceptable toxicity (Phase 1b). The primary objective was to assess safety/tolerability of ramucirumab in combination with durvalumab; preliminary efficacy will be examined in expansion cohorts.

As of the data cutoff on Jun. 27, 2016, a total of 20 patients were treated in phase 1a; NSCLC and G/GEJ DLT observation is completed. One G/GEJ patient in the phase 1a had confirmed partial response (cycle 2) resulting in an ORR of 14.3%. The patient had a −41.89% tumor percentage change from baseline after the DLT period, at the time of the data cutoff.

As of the data cut-off Dec. 12, 2016, 26 G/GEJ patients were treated. The median age was 55, 73% male, 65% ECOG PS 1. Median duration on treatment was 2.30 months for ramucirumab and 2.43 months for durvalumab, where 13 patients remain on treatment. Treatment-emergent AEs (TEAE) occurred in 23 (88%) patients, most commonly headache (38%), fatigue (38%), hypertension (35%), diarrhea (35%), nausea (35%), abdominal pain (31%), vomiting (31%), decreased appetite (27%), and pyrexia (27%). 13 (50%) patients experienced TEAE≥grade 3. Treatment-related adverse events occurred in 17 (65%) patients, most commonly hypertension (31%), headache (27%), diarrhea (23%), fatigue (23%), and pyrexia (12%) (no febrile neutropenia). Five (19%) patients experienced grade 3 TRAEs (proteinuria n=1, hypertension n=4). No treatment-related grade 4 or 5 events occurred. Six (23%) patients experienced serious adverse events, 2 (7%) pts experienced SAE related to treatment (Gr 2 diarrhea n=1, grade 3 proteinuria n=1). Four (15%) patients had a partial response, 8 (31%) patients had stable disease, 10 (38%) patients had progressive disease, and 4 (15%) patients were non-evaluable. The disease control rate was 46% (12/26). Overall response rate was 15%, with a median time to response of 1.46 months. Three of the four responders remain on treatment. The PK profiles of ramucirumab and durvalumab were typical for an IgG1 mAb. PD-L1 expression status and other genetic factors associated with gastric cancer are being assessed.

SEQUENCE LISTING

(Anti-Human VEGFR-2 Antibody, LCVR) (Artificial
Sequence)
SEQ ID NO: 1
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIK
(Anti-Human VEGFR-2 Antibody, HCVR) (Artificial
Sequence)
SEQ ID NO: 2
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSS
(Anti-Human VEGFR-2 Antibody, LC) (Artificial
Sequence)
SEQ ID NO: 3
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
(Anti-Human VEGFR-2 Antibody, LC) (Artificial
Sequence)
SEQ ID NO: 4
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSSASTKGPSVLPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
(Anti-Human PD-L1 Antibody, LCVR) (Artificial
Sequence)
SEQ ID NO: 5
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK
(Anti-Human PD-Ll Antibody, HCVR) (Artificial
Sequence)
SEQ ID NO: 6
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS
(Anti-Human PD-L1 Antibody, LC) (Artificial
Sequence)
SEQ ID NO: 7
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
(Anti-Human PD-L1 Antibody, HC) (Artificial
Sequence)
SEQ ID NO: 8
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K
(Human VEGFR-2) (Homo Sapiens)
SEQ ID NO: 9
MQSKVLLAVALWLCVETRAASVGLPSVSLDLPRLSIQKDILTIKANTTLQ
ITCRGQRDLDWLWPNNQSGSEQRVEVTECSDGLFCKTLTIPKVIGNDTGA
YKCFYRETDLASVIYVYVQDYRSPFIASVSDQHGVVYITENKNKTVVIPC
LGSISNLNVSLCARYPEKRFVPDGNRISWDSKKGFTIPSYMISYAGMVFC
EAKINDESYQSIMYIVVVVGYRIYDVVLSPSHGIELSVGEKLVLNCTART
ELNVGIDFNWEYPSSKHQHKKLVNRDLKTQSGSEMKKFLSTLTIDGVTRS
DQGLYTCAASSGLMTKKNSTFVRVHEKPFVAFGSGMESLVEATVGERVRI
PAKYLGYPPPEIKWYKNGIPLESNHTIKAGHVLTIMEVSERDTGNYTVIL
TNPISKEKQSHVVSLVVYVPPQIGEKSLISPVDSYQYGTTQTLTCTVYAI
PPPHHIHWYWQLEEECANEPSQAVSVTNPYPCEEWRSVEDFQGGNKIEVN
KNQFALIEGKNKTVSTLVIQAANVSALYKCEAVNKVGRGERVISFHVTRG
PEITLQPDMQPTEQESVSLWCTADRSTFENLTWYKLGPQPLPIHVGELPT
PVCKNLDTLWKLNATMFSNSTNDILIMELKNASLQDQGDYVCLAQDRKTK
KRHCVVRQLTVLERVAPTITGNLENQTTSIGESIEVSCTASGNPPPQIMW
FKDNETLVEDSGIVLKDGNRNLTIRRVRKEDEGLYTCQACSVLGCAKVEA
FFIIEGAQEKTNLEIIILVGTAVIAMFFWLLLVIILRTVKRANGGELKTG
YLSIVMDPDELPLDEHCERLPYDASKWEFPRDRLKLGKPLGRGAFGQVIE
ADAFGIDKTATCRTVAVKMLKEGATHSEHRALMSELKILIHIGHHLNVVN
LLGACTKPGGPLMVIVEFCKFGNLSTYLRSKRNEFVPYKTKGARFRQGKD
YVGAIPVDLKRRLDSITSSQSSASSGFVEEKSLSDVEEEEAPEDLYKDFL
TLEHLICYSFQVAKGMEFLASRKCIHRDLAARNILLSEKNVVKICDFGLA
RDIYKDPDYVRKGDARLPLKWMAPETIFDRVYTIQSDVWSFGVLLWEIFS
LGASPYPGVKIDEEFCRRLKEGTRMRAPDYTTPEMYQTMLDCWHGEPSQR
PTFSELVEHLGNLLQANAQQDGKDYIVLPISETLSMEEDSGLSLPTSPVS
CMEEEEVCDPKFHYDNTAGISQYLQNSKRKSRPVSVKTFEDIPLEEPEVK
VIPDDNQTDSGMVLASEELKTLEDRTKLSPSFGGMVPSKSRESVASEGSN
QTSGYQSGYHSDDTDTTVYSSEEAELLKLIEIGVQTGSTAQILQPDSGTT
LSSPPV
(Human PD-L1) (Homo Sapiens)
SEQ ID NO: 10
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET

1. A method of treating advanced gastric or gastroesophageal junction adenocarcinoma, non-small cell lung cancer, or hepatocellular carcinoma comprising administering to a patient in need thereof, an effective amount of an anti-human VEGFR-2 (SEQ ID NO: 9) antibody comprising a light chain variable region having the amino acid sequence of SEQ ID NO: 1 and a heavy chain variable region having the amino acid sequence of SEQ ID NO: 2 in simultaneous, separate, or sequential combination with an effective amount of an anti-human PD-L1 (SEQ ID NO: 10) antibody comprising a light chain variable region having the amino acid sequence of SEQ ID NO: 5 and a heavy chain variable region having the amino acid sequence of SEQ ID NO: 6; wherein (a) the anti-human VEGFR-2 antibody is administered at a dose of 8 mg/kg every two weeks and the anti-human PD-L1 antibody is administered at a dose of 750 mg every two weeks when advanced gastric or gastroesophageal junction adenocarcinoma or hepatocellular carcinoma is treated, or (b) the anti-human VEGFR-2 antibody is administered at a dose of 10 mg/kg every three weeks and the anti-human PD-L1 antibody is administered at a dose of 1125 mg every three weeks when non-small cell lung cancer is treated.

2. The method of claim 1, wherein the anti-human VEGFR-2 antibody further comprises a light chain having the amino acid sequence of SEQ ID NO: 3 and a heavy chain having the amino acid sequence of SEQ ID NO: 4.

3. The method of claim 1, wherein the anti-human PD-L1 antibody further comprises a light chain having the amino acid sequence of SEQ ID NO: 7 and a heavy chain having the amino acid sequence of SEQ ID NO: 8.

4. The method of claim 1, wherein advanced gastric or gastroesophageal junction adenocarcinoma is treated and the anti-human VEGFR-2 antibody is administered at a dose of 8 mg/kg every two weeks and the anti-human PD-L1 antibody is administered at a dose of 750 mg every two weeks.

5. The method of claim 1, wherein non-small cell lung cancer is treated and the anti-human VEGFR-2 antibody is administered at a dose of 10 mg/kg every three weeks and the anti-human PD-L1 antibody is administered at a dose of 1125 mg every three weeks.

6. The method of claim 1, wherein hepatocellular carcinoma is treated and the anti-human VEGFR-2 antibody is administered at a dose of 8 mg/kg every two weeks and the anti-human PD-L1 antibody is administered at a dose of 750 mg every two weeks.

7. A kit for the treatment of advanced gastric or gastroesophageal junction adenocarcinoma, non-small cell lung cancer, or hepatocellular carcinoma, the kit comprising an anti-human VEGFR-2 (SEQ ID NO: 9) antibody comprising a light chain having the amino acid sequence of SEQ ID NO: 3 and a heavy chain having the amino acid sequence of SEQ ID NO: 4 and an anti-human PD-L1 (SEQ ID NO: 10) antibody comprising a light chain having the amino acid sequence of SEQ ID NO: 7 and a heavy chain having the amino acid sequence of SEQ ID NO: 8.

8. A kit for the treatment of advanced gastric or gastroesophageal junction adenocarcinoma, non-small cell lung cancer, or hepatocellular carcinoma, the kit comprising a first pharmaceutical composition comprising an anti-human VEGFR-2 (SEQ ID NO: 9) antibody comprising a light chain having the amino acid sequence of SEQ ID NO: 3 and a heavy chain having the amino acid sequence of SEQ ID NO: 4 and a second pharmaceutical composition comprising an anti-human PD-L1 (SEQ ID NO: 10) antibody comprising a light chain having the amino acid sequence of SEQ ID NO: 7 and a heavy chain having the amino acid sequence of SEQ ID NO: 8.

9. The kit of any one of claim 7 or 8, wherein the first pharmaceutical composition further comprises one or more pharmaceutically acceptable carriers, diluents, or excipients, and the second pharmaceutical composition further comprises one or more pharmaceutically acceptable carriers, diluents or excipients.

10.-27. (canceled)
